(19) United States
(12) Patent Application Publication
(10) Pub. No.: US 2021/0298833 A1
(43) Pub. Date: Sep. 30, 2021

(54) NAVIGATION AUTO FOCUS SYSTEM

(71) Applicant: Mitaka Kohki Co., Ltd., Tokyo (JP)

(72) Inventor: Katsushige NAKAMURA, Tokyo (JP)

(73) Assignee: Mitaka Kohki Co., Ltd., Tokyo (JP)

(21) Appl. No.: 17/186,453

(22) Filed: Feb. 26, 2021

(30) Foreign Application Priority Data
Mar. 30, 2020 (JP) 2020-059680
Jun. 29, 2020 (JP) 2020-111353

(51) Int. Cl. A61B 34/20; A61B 1/00

(52) U.S. Cl. CPC A61B 34/20; A61B 2034/2068; A61B 2034/2055; A61B 1/00149

(57) ABSTRACT

A controller calculates a focal length of an objective optical system on the basis of position information acquired by a position detector and outputs to a focusing unit a signal for moving a movable lens in a focus direction to make the focus of the objective optical system coincide with a focus point specified by a pointer. It is not necessary to equip an autofocus system to a surgical microscope, and automatic focusing is performed by using a navigation system.

### BACKGROUND OF THE INVENTION

The present invention relates to a navigation auto focus system.

A medical optical device, such as a surgical microscope or a camera, is used when a doctor performs an operation in neurosurgery or the like while magnifying and observing a surgical site. The medical optical device is supported at the tip of an arm of a stand device. Changing the position and orientation of the medical optical device enables the surgical site to be observed and captured from all directions.

The medical optical device is provided with an autofocus system. There are various types of autofocus systems, such as a system receiving a part of a luminous flux in a medical optical device by an image sensor and controlling on the basis of its contrast, and a system catching scanning light (laser) reflected from an object by an image sensor and controlling on the basis of its deviation. With the autofocus system, a surgical site being observed or captured can be automatically focused though the medical optical device is in any position and orientation.

In contrast, there is also a known navigation system using a pointer capable of indicating a position to be observed, detecting by a position detector of navigation the position of a marker attached to each of the pointer and a medical optical device, and thus selecting, from medical information image data (CT, MRI), an image corresponding to the specified position of the tip of the pointer and having a directionality corresponding to the position and orientation of the medical optical device at that time, and displaying the selected image on a monitor together with the tip of the pointer. Earlier technology is disclosed in U.S. Pat. No. 9,392,931 (Patent Literature 1).

### SUMMARY OF THE INVENTION

However, in such earlier technology, the autofocus system and the navigation system are provided on the medical optical device independently of each other. To perform automatic focusing for the medical optical device, the medical optical device includes the autofocus system to always perform autofocus control, and automatic focusing cannot be performed without the autofocus system.

In response to the above issue, it is an object of the present invention to provide a navigation autofocus system for a medical optical device that performs autofocus for the medical optical device using a navigation system without providing the medical optical device with an autofocus system.

According to a technical aspect of the present invention, a navigation autofocus system for a medical optical device includes (i) a medical optical device configured to be supported at a tip of an arm of a stand device, (ii) a pointer configured to be held by hand to indicate a focus point, (iii) a switcher configured to specify the focus point indicated by the pointer, (iv) a marker attached to each of the medical optical device and the pointer, (v) a position detector configured to acquire position information related to a position and an orientation of the medical optical device and the focus point specified by detecting the marker, and (v) a controller configured to calculate the focal length of the objective optical system on the basis of the position information acquired by the position detector and control the focusing unit to move a movable lens in a focus direction to make the focus of the objective optical system coincide with the focus point specified on the basis of the focal length calculated. The medical optical device is supported at the tip of the arm of the stand device to be movable to any position and any orientation, and includes at least an objective optical system and a focusing unit, the objective optical system including a movable lens for varying a focal length, the movable lens being movable along an optical axis, the focusing unit being for moving the movable lens, the medical optical device being configured to enlarge and to observe or image an area including a focus point corresponding to the focal length.

### BRIEF DESCRIPTION OF THE DRAWING

FIG. 1 is an overall view of a surgical microscope supported by a stand device according to a first embodiment.

FIG. 2 is a diagram illustrating the structure of the surgical microscope.

FIG. 3 is a diagram illustrating autofocus states of the surgical microscope.

FIG. 4 is an overall view of a surgical microscope supported by a stand device according to a second embodiment.

### DESCRIPTION OF THE PREFERRED EMBODIMENTS

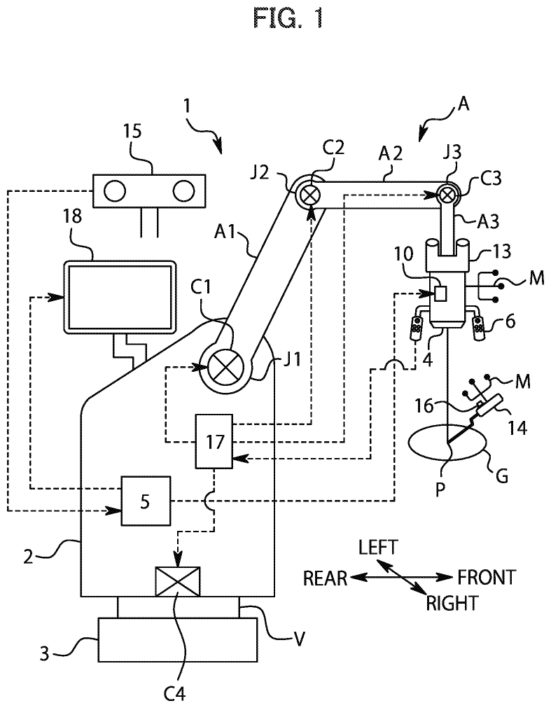

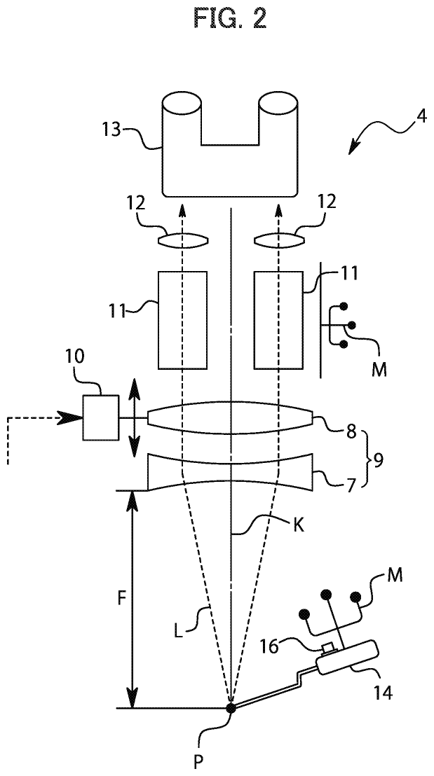

FIGS. 1 to 3 illustrate a first embodiment of the present invention. The longitudinal and lateral directions are illustrated as in FIG. 1.

A stand device 1 installed in an operating room basically includes a stand body 2 and an arm A. The stand body 2 is installed on a base 3, and the whole is rotatable on a vertical shaft V with respect to the base 3 in the horizontal direction. Provided on the vertical shaft V of the base 3 is a rotary driving unit C4. Supported at the tip of the arm A as a medical optical device is a surgical microscope 4.

The stand body 2 has a monitor 18 mounted thereon. The monitor 18 is for displaying medical information images, such as CT, and MRI. The monitor 18 is connected to a controller 5 described later.

The arm A includes a first arm portion A1, a second arm portion A2, and a third arm portion A3. The first arm portion A1 closest to the base end has its base end rotatably supported to the stand body 2 by use of a first joint J1. The second arm portion A2 is rotatably supported to the first arm portion A1 by a second joint J2. The third arm portion A3 is rotatably supported to the second arm portion A2 by a third joint J3.

The first to third joints J1 to J3 of the arm A are provided with rotary drive units C1 to C3, respectively. The rotary drive units C1 to C3 and the rotary drive unit C4 of the vertical shaft V are connected to an operation unit 17 in the stand body 2 and are configured to electrically rotate by an actuator in response to a signal from the operation unit 17. A joint portion not shown is provided at the lower end of the third arm portion A3. The joint portion can tilt the surgical microscope 4 back, forth, left, and right and is also provided with a rotary drive unit. The joint portion (not shown), the first to third joints J1 to J3, and the vertical shaft V are rotatable, and thus the position and orientation of the surgical microscope 4 supported by the tip of the arm A are arbitrarily changed.

The surgical microscope 4 is provided with a handle 6 on both right and left sides thereof. The handle 6 is also connected to the operation unit 17. The handle 6 is provided with multiple buttons and a joystick. By operating the buttons and joystick, a signal for instructing or changing the position and orientation of the surgical microscope 4 is outputted to the operation unit 17.

The surgical microscope 4 is provided with an objective optical system 9 comprising a fixed lens 7 and a movable lens 8 therein. The movable lens 8 is movable along an optical axis K by a focusing unit 10. By moving the movable lens 8, a focal length F of the objective optical system 9 is changed. The focusing unit 10 is connected to the controller 5 and moves the movable lens 8 in response to a signal from the controller 5.

As a focal point (F) on the optical axis K agrees with a focus point P specified by a marker M of a pointer 14, reflected light L from the focus point P passes through the objective optical system 9, a pair of right and left variable power optical systems 11, and imaging lenses 12 to be guided to an eyepiece portion 13.

The surgical microscope 4 has a marker M for navigation attached thereto. Reference numeral 14 denotes a handy pointer for navigation, and the pointer 14 is also provided with a marker M. The pointer 14 is handheld and for indicating by its tip the focus point P to be focused in a surgical site G.

The operating room is provided with a position detector 15 for navigation. The position detector 15 acquires position information related to the position and orientation of the surgical microscope 4 and the tip position of the pointer 14 by detecting each marker M. The position information acquired by the position detector 15 is sent to the controller 5.

The pointer 14 has a switcher 16 such as a button switch, and the tip position of the pointer 14 is specified as the focus point P when the switcher 16 is operative. The position information of the specified focus point P is sent to the controller 5.

The operation is explained. First, when it is desired to set the surgical microscope 4 to an arbitrary position and orientation, a button or the like of the handle 6 of the surgical microscope 4 is operated to output to the operation unit 17 a signal related to the desired direction and orientation to which the surgical microscope 4 is moved. Then, the operation unit 17 sends a signal to each of the rotary driving units C1 to C4 and the like. The rotary driving units C1 to C4 and the like rotate to move the surgical microscope 4 to a position and an orientation in which a desired visual field is obtained in the surgical site G. The surgical microscope 4 is electrically moved and easily operated by a doctor without using a large force.

Next, a position where focusing is needed in the surgical site G in the surgical microscope 4 is pointed by the tip of the pointer 14, and the switcher 16 of the pointer 14 is operated at the position. Then, the tip position information acquired by the position detector 15 at that time is sent to the controller 5 as the position associated with the focus point P.

When the focus point P is specified, the controller 5 calculates the focal length F of the objective optical system 9 from each acquired position information of the surgical microscope 4 and the focus point P and outputs to the focusing unit 10 a signal for moving the movable lens 8 in a focus direction to focus on the specified focus point P. Thus, the focus point Pin the surgical site G can be clearly observed in a state where the focus of the objective optical system 9 coincides with the specified focus point P.

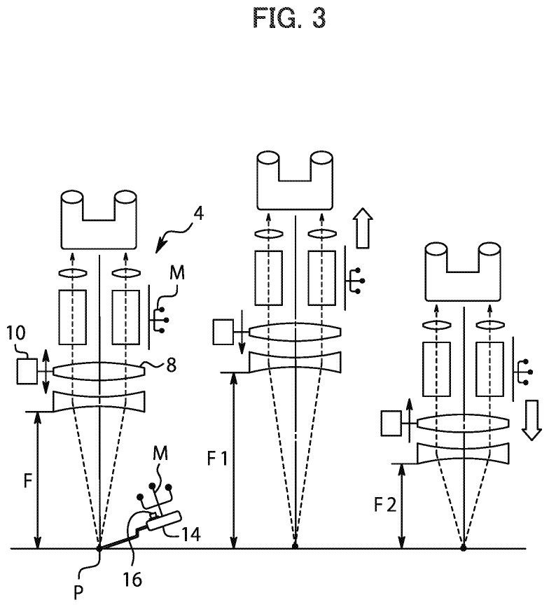

Once the specified focus point P is brought into focus, when the position of the surgical microscope 4 is later moved up and down (or tilt) as illustrated in FIG. 3, the controller 5 automatically calculates focal lengths F1, F2 and continues to control the focusing unit 10 in response thereto, so that the focusing state of the focus point P is maintained

When the focus point P is specified, an image corresponding to the specified focus point P and having a directivity corresponding to the position and orientation of the surgical microscope 4 is selected from medical information image data (CT, MRI), and the selected image is displayed on the monitor 18 together with the focus point P. Accordingly, the doctor can confirm the situation around the focus point P by looking at the monitor 18 and perform the operation more accurately.

According to the present embodiment, as described above, automatic focusing is performed by using a navigation system without providing an autofocus system in the surgical microscope 4.

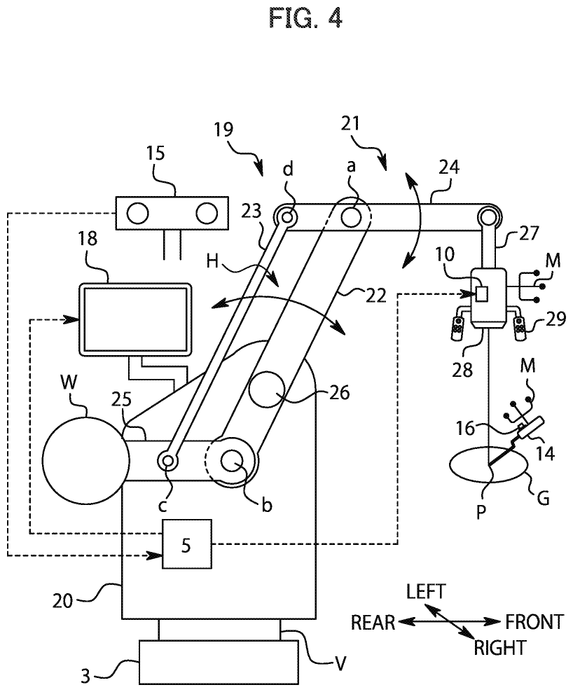

FIG. 4 is a diagram illustrating a second embodiment of the present invention. The present embodiment includes components similar to those in the first embodiment. The similar components are thus denoted by the same reference numerals, and the redundant description is omitted.

A stand body 20 of a stand device 19 is manually rotatable on the vertical shaft V with respect to the base 3 in the horizontal direction.

The stand body 20 is provided with an arm 21 of a weight balanced type. The arm 21 includes a parallel linkage H (a-b-c-d-a) having a structure in which a front arm portion 22, a rear arm portion 23, an upper arm portion 24, and a lower arm portion 25 are rotatably supported at four connecting portions a, b, c, d, respectively. The parallel linkage H is attached to the stand body 20 rotatably as a whole on a rotary shaft 26 fixed in the middle of the front arm portion 22.

The upper arm portion 24 extends forward beyond the parallel linkage H and is vertically rotatable about the connecting portion a that links with the front arm portion 22. As the upper arm portion 24 is rotated, the lower arm portion 25 is also rotated due to the parallel linkage H in a manner that the upper arm portion 24 and the lower arm portion 25 are always parallel.

The upper arm portion 24 has a tip arm portion 27 attached at the front end part thereof. The tip arm portion 27 is associated with a mechanism, not shown, provided in the upper arm portion 24 at a connection portion connecting with the upper arm portion 24, and the tip arm portion 27 is configured to always maintain a vertical position regardless of how the upper arm portion 24 is rotated up and down.

The tip arm portion 27 has a camera 28 as a medical optical device supported at the lower end part thereof. The camera 28 has a marker M attached thereto and is provided with the focusing unit 10 therein for changing the focal length.

The tip arm portion 27 has a joint portion, not shown, provided at the lower end part thereof. The joint portion enables the camera 28 to be tilted in front, rear, left and right directions with respect to the lower end part of the tip arm portion 27. The camera 28 takes a three-dimensional image of the surgical site G including the focus point P and displays the image on the monitor 18. A handle 29 is provided on both right and left sides of the camera 28. The handle 29 is provided with multiple buttons and a joystick.

The lower arm portion 25 extends rearward beyond the parallel linkage H and has a counterweight W attached to the rear end part thereof that is for balancing the weight around the rotary shaft 26.

The rotary shaft 26 and the upper and lower connecting portions a, b of the front arm portion 22, and the joint portion (not shown) of the lower end part of the tip arm portion 27 are each provided with a clutch mechanism. The clutch mechanism is turned off (rotation free) by operating a button or the like provided on the handle 29 of the camera 28 with a finger and turned on (rotation locked) by release the finger from the button or the like.

Accordingly, the doctor manually moves the arm 21 freely to set the camera 28 in a desired position and orientation by grasping the handle 29 by hand and operating the button or the like with his finger to allow the connecting portions a, b of the arm 21 to be rotatable. At this time, the weight of the arm 21 is balanced, and thus the doctor can easily move the arm 21 with a small operating force.

Then, when the camera 28 is set at a desired position and orientation to capture the surgical site G and then the doctor releases his finger from the button or the like of the handle 29, rotation of the connecting portions a, b of the arm 21 and the like are locked, and the state is maintained.

When the focus point P is specified by the pointer 14, the controller 5 calculates the focal length on the basis of each position information of the camera 28 and the focus point P as in the previous embodiment, and outputs a signal to the focusing unit 10 to bring the specified focus point P into focus. As results, the specified focus point P is brought into a focused state, and the focus point P is clearly captured. Then, when the camera 28 is moved up and down or tilted again by holding the handle 29, the focus point P remains in focus.

The image captured by the camera 28 is displayed on the monitor 18, and the doctor can observe the focus point P in the surgical site G in three dimensions by viewing the monitor 18 through the dedicated 3D glasses.

The monitor 18 can display medical information images related to the focus point P overlaid on the top of the image captured by the camera 28 or can display them in parallel.

According to a technical aspect of the present invention, the controller calculates a focal length of the objective optical system on the basis of position information acquired by the position detector and outputs a signal for moving the movable lens in a focus direction with respect to the focusing unit to make the focus of the objective optical system coincide with the focus point specified by the pointer. Accordingly, it is not necessary to equip a conventional autofocus system having a complicated structure and requiring constant feedback control in the medical optical device, and automatic focusing is performed using the navigation system.

According to a technical aspect of the present invention, the rotary drive units are provided at the joint of the arm for supporting the medical optical device, and the position and orientation of the medical optical device are changed by electric power by controlling the rotary drive units by the operation unit.

According to another technical aspect of the present invention, the arm attached to the stand body is a balance type including the parallel linkage, and thus the arm is manually moved to change the position and orientation of the medical optical device. Since the weight is balanced, the manual operation of the arm needs less force and is easy.

This application claims benefit of priority under 35 USC § 119 to Japanese Patent Applications No. 2020-59680 filed on Mar. 30, 2020 and No. 2020-111353 filed on Jun. 29, 2020, the entire content of which is incorporated by reference herein.

What is claimed is:

1. A navigation autofocus system for a medical optical device, comprising:
a medical optical device configured to be supported at a tip of an arm of a stand device to be movable to any position and any orientation, the medical optical device comprising at least an objective optical system and a focusing unit, the objective optical system including a movable lens for varying a focal length, the movable lens being movable along an optical axis, the focusing unit being for moving the movable lens, and the medical optical device being configured to enlarge and to observe or image an area including a focus point corresponding to the focal length;
a handheld pointer for indicating the focus point;
a switcher configured to specify the focus point indicated by the pointer;
a marker attached to each of the medical optical device and the pointer;
a position detector configured to detect the marker and acquire position information related to a position and an orientation of the medical optical device and the focus point specified by the marker; and
a controller for calculating the focal length of the objective optical system on the basis of the position information acquired by the position detector and controlling the focusing unit to move the movable lens in a focus direction to make the focus of the objective optical system coincide with the focus point specified on the basis of the calculated focal length.

2. The navigation autofocus system for the medical optical device according to claim 1, wherein
the stand device comprises a stand body installed on a floor, and an arm including a plurality of arm portions linked via rotatable joints;
an arm portion of the plurality of arm portions being closest to a base end thereof is attached to the stand body via one of the joints;
each of the joints of the arm is provided with a rotary drive unit; and
each of the joints is connected to an operation unit, and the operation unit outputs a rotation signal to each rotary drive unit to electrically change the position and orientation of the medical optical device supported at the tip of the arm.

3. The navigation autofocus system for the medical optical device according to claim 1, wherein
the stand device having a stand body installed on a floor; and an arm in a wight balanced type comprising a plurality of arm portions combined to configure a parallel linkage;
the arm has the parallel linkage attached to the stand body in a rotatable manner on a rotary shaft set in the middle of the front arm portion, the parallel linkage including a front arm portion and a rear arm portion in parallel, and an upper arm portion and a lower arm portion in parallel, connecting portions of the front, rear, upper, lower arm portions being rotatably supported;
the upper arm portion extends forward beyond the parallel linkage and has a tip arm portion connected to a front end part thereof, the tip arm portion being always maintained in a vertical state, and the medical optical device being supported at a lower end part of the tip arm portion; and
the lower arm portion extends rearward beyond the parallel linkage and has a counter weight supported at a rear end part thereof, the counter weight being for balancing a weight around the rotary shaft.
